(19) United States
(12) Patent Application Publication
(10) Pub. No.: US 2025/0228758 A1
(43) Pub. Date: Jul. 17, 2025

(54) Solid Cleansing Compositions

(71) Applicant: KENVUE BRANDS LLC, Summit, NJ (US)

(72) Inventor: Tatiana Luts, Bridgewater, NJ (US)

(21) Appl. No.: 18/700,453

(22) PCT Filed: Oct. 26, 2023
(86) PCT No.: PCT/IB2023/060815

§ 371 Date: Apr. 11, 2024

Related U.S. Application Data
(60) Provisional application No. 63/419,847, filed on Oct. 27, 2022.

(51) Int. Cl. A61K 8/44; A61K 8/02; A61K 8/23; A61K 8/365; A61K 8/46; A61Q 19/10

(52) U.S. Cl. CPC A61K 8/447; A61K 8/022; A61K 8/23; A61K 8/365; A61K 8/466; A61Q 19/10

(57) ABSTRACT

Solid cleansing compositions including at least a first surfactant and a second surfactant in which the combined weight of the first surfactant and the second surfactant is at least about 60% by weight, based on the total weight of the solid cleansing composition. The solid cleansing composition can be in a powder form.

### 1. FIELD

The present disclosure relates to improved cleansing compositions, and in particular solid cleansing compositions. Such cleansing compositions are substantially free of sulfate surfactants, dissolvable in the presence of a fluid such as water, and in some aspects can be in the form of a powder. In certain aspects, the cleansing compositions of the present disclosure can be combined with a fluid such as water and be reconstituted prior to use.

### 2. BACKGROUND

Cleansing products are available in various forms including liquid forms. Liquid cleansers contain a significant amount of water (typically greater than 70%), are susceptible to leaks and spills, and typically require larger packaging such as bottles, tubes, and jars. As such, liquid cleansers can cause a higher negative environmental impact when volumes of them are shipped worldwide and can be less convenient for a user. In certain instances, it is therefore preferable to utilize a solid cleansing product which is dissolvable and reconstitutable in the presence of a fluid such as water. Such solid cleansers can be more environmentally conscious through the use of less plastic since they can be sold in smaller packaging such as sachets, packets, stick packs, and pouches. Additionally, solid cleansers can consume less energy during their manufacturing process as fewer batches are required to supply the demand and the process typically does not require heat. However, solid cleansing products can also be challenging to formulate and manufacture, for example, since they require ingredients of a specific solid form which are also easily blendable into a final uniform product.

There is therefore a need for improved solid cleansing products, particularly in the form of a powder, which are sufficiently mild to use on the skin of the user, which are dissolvable in the presence of a fluid such as water, and which deliver desired cleansing levels without harshness, dryness or irritation to the user's skin. There is also a need for solid cleansing products which provide a reduced environmental impact compared to other cleansing product forms. The present disclosure addresses these and other needs.

### 3. SUMMARY

The presently disclosed subject matter provides solid cleansing compositions. The solid cleansing compositions include at least a first surfactant and at least a second surfactant. The first surfactant is present in a first surfactant weight amount. The second surfactant is present in a second surfactant weight amount. The combined weight of the first surfactant and the second surfactant is at least about 60% by weight, based on the total weight of the solid cleansing composition. The solid cleansing composition is in a powder form.

In certain embodiments, the combined weight of the first surfactant and the second surfactant is from about 60% to about 90% by weight, based on the total weight of the solid cleansing composition.

In certain embodiments, the first surfactant weight amount and the second surfactant weight amount can be approximately equal to each other.

In certain embodiments, the first surfactant and the second surfactant can have the same ionic nature.

In certain embodiments, the first surfactant and the second surfactant can be selected from anionic surfactants, nonionic surfactants, cationic surfactants, or amphoteric surfactants.

In certain embodiments, the first surfactant can be a taurate.

In certain embodiments, the second surfactant can be an isethionate.

In certain embodiments, the solid cleansing composition can be substantially free of sulfate surfactants.

In certain embodiments, the solid cleansing composition can further include one or more pH adjusters comprising citric acid, magnesium sulfate, or combinations thereof.

In certain embodiments, the solid cleansing composition can be dissolvable in a liquid.

The presently disclosed subject matter further provides a kit. The kit includes a solid cleansing composition and a container. The solid cleansing composition includes at least a first surfactant and at least a second surfactant. The combined weight of the first surfactant and the second surfactant is at least about 60% by weight, based on the total weight of the solid cleansing composition. The solid cleansing composition is in a powder form. The container is configured to contain a liquid.

The presently disclosed subject matter further provides a cleansing product. The cleansing product includes from about 2% to about 20% by weight of a solid cleansing composition and from about 80% to about 98% by weight of a liquid, based on the total weight of the cleansing product. The solid cleansing composition includes at least a first surfactant and at least a second surfactant. The combined weight of the first surfactant and the second surfactant is at least about 60% by weight, based on the total weight of the solid cleansing composition. The solid cleansing composition is in a powder form. The cleansing composition is dissolved in the liquid.

### 4. DETAILED DESCRIPTION

The presently disclosed subject matter relates to improved solid cleansing compositions which advantageously provide desired cleansing levels to a user. Such solid cleansing compositions can have a surfactant content of at least about 60% by weight, based on the total weight of the solid cleansing composition. In certain aspects, the solid cleansing compositions of the present disclosure can be in the form of a powder.

These and other aspects of the disclosed subject matter are discussed in more detail below and in the Examples. For clarity, and not by way of limitation, this detailed description is divided into the following sub-portions:
4.1 Definitions;
4.2 Cleansing Compositions;
4.3 Methods of Making Cleansing Compositions;
4.4 Methods of Using Cleansing Compositions; and 4.5 Features of Cleansing Compositions.

### 4.1. Definitions

The terms used in this specification generally have their ordinary meanings in the art within the context of this disclosure and in specific context where each term is used. Certain terms are discussed below, or elsewhere in the specification, to provide additional guidance in describing the compositions and methods of the disclosure and how to make and use them.

As used in the specification and the appended claims, the singular forms “a,” “an,” and “the” include the plural referents unless the context clearly dictates otherwise. Thus, for example, reference to “an ingredient” includes mixtures of ingredients.

As used herein, the term “about” means within an acceptable error range for the particular value as determined by one of ordinary skill in the art, which will depend in part on how the value is measured or determined (i.e., the limitations of the measurement system). For example, “about” can mean within three or more standard deviations per practice in the art. Alternatively, “about” can mean a range of up to 10%, up to 5%, up to 3%, up to 1%, or up to 0.5% of a given value. Particularly with respect to systems or processes, the term can mean within an order of magnitude, for example, within five-fold or within two-fold of a value.

As used herein, the terms “comprises,” “comprising,” or any other variation thereof, are intended to cover a non-exclusive inclusion, such that a process, method, article, or apparatus that comprises a list of elements does not include only those elements but can include other elements not expressly listed or inherent to such process, method, article, or apparatus.

As used herein, the term “powder” refers to a particulate material.

As used herein, the term “solid” refers to a material that is not in liquid form. Solid materials can include flowable granules or powders. In certain aspects, solid materials as described herein can be anhydrous in nature. The cleansing compositions herein can be substantially free or free of added water.

As used herein, the term “substantially free” refers to a composition which contains less than about 0.1% by weight or less than about 0.01% by weight of a component or ingredient.

As used herein, the term “substantially free of added water” refers to the cleansing composition having less than about 3.5%, less than about 2%, less than about 1.5%, less than about 1%, less than about 0.5%, less than about 0.1%, or less than about 0.01% of added water. The cleansing compositions free of added water can include trace levels of water, for example, due to the presence of water in ingredients such as fragrances, extracts, or the like, or due to the presence of moisture in the air. In certain embodiments, the amount of trace water in the cleansing composition can be less than about 3.5%, less than about 2%, less than about 1.5%, less than about 1%, less than about 0.5%, less than about 0.1%, or less about 0.01% by weight, based on the total weight of the cleansing composition.

As used herein, the terms “% w/w” or “weight percent” refers to the percentage of an ingredient(s)/the total percentage by weight of the composition (100%). The terms “% w/w” or “weight percent” refer to the quantity by weight of a constituent or component. The terms “weight percent”, “wt-%”, “wt. %”, and “wt %” are used interchangeably.

### 4.2. Cleansing Compositions

Cleansing compositions of the present disclosure advantageously provide a solid cleansing composition with desired cleansing properties. Such cleansing compositions can include one or more surfactants, one or more chelating agents, one or more pH adjusters, one or more preservatives, one or more antioxidants, one or more additional additives, or combinations thereof. The one or more surfactants can be present in an amount of at least 60% by weight, based on the total weight of the solid cleansing composition.

By way of example and not limitation, the one or more additional additives can include one or more colorants, one or more fragrances, one or more bulking/anti-caking agents, one or more skin benefit agents, one or more thickeners, one or more effervescent agents, and combinations thereof.

### 4.2.1. Surfactants

The cleansing composition can include one or more surfactants. In certain embodiments, the cleansing composition can include at least two surfactants, for example, in which the first surfactant and the second surfactant are different from each other.

Surfactants suitable for use with the present disclosure include anionic surfactants, such as sulfonates (olefin sulfonates, isethionates, taurates, sulfoacetates, sulfosuccinates, sulfolaurates), aminoacid-type surfactants (glycinates, glutamates and sarcosinates), crypto anionic surfactant (phosphates, carboxylates, citrates), polymeric surfactants (Sodium Hydrolyzed Potato Starch Dodecenylsuccinate, Potassium Acrylates Copolymer) and combinations thereof. Further surfactants suitable for use with the present disclosure include cationic surfactants, such as quaternary ammonium salts, amine oxides, and ester quats; amphoteric surfactants, such as betaines, amidobetaines, ester betaines, amphoacetates, ampho diacetates, sultaines; and nonionic surfactants, such as alkyl polyglucosides, poloxamers, polysorbates, ethoxylated fatty alcohols, ethoxylated fatty acids, ethoxylated sorbitan esters, ethoxylated glyceryl esters, polyglyceryl esters.

In certain embodiments, the cleansing composition can include at least two or more surfactants. The two or more surfactants can both be anionic surfactants, nonionic surfactants, cationic surfactants, or amphoteric surfactants. In certain embodiments, the first surfactant and the second surfactant can be the same in ionic nature. For example, and not by way of limitation, in certain embodiments, the cleansing composition can include a first surfactant and a second surfactant. The first surfactant and the second surfactant can both be anionic surfactants, e.g., the first surfactant can be sodium methyl cocoyl taurate and the second surfactant can be sodium cocoyl isethionate.

Surfactants can be present in an amount of at least about 60%, at least about 65%, at least about 70%, at least about 75%, at least about 80%, at least about 85%, or at least about 90% by weight, based on the total weight of the cleansing composition. In certain embodiments surfactants can be present in an amount of from about 60% to about 90%, about 60% to about 75%, or about 65% to about 85% by weight, based on the total weight of the cleansing composition. In particular embodiments, the cleansing composition can include surfactants in an amount of about 60%, about 61%, about 65%, about 70%, about 75%, about 80%, about 85%, or about 90% by weight, based on the total weight of the cleansing composition.

If more than one surfactant is used, each surfactant may be present in the same weight amount or may be present in different weight amounts. In certain embodiment, the first surfactant and the second surfactant can be present in weight amounts approximately equal to each other. In certain embodiments, the first surfactant can be present in an amount of at least about 15%, at least about 20%, at least about 25%, or at least about 30% by weight, based on the total weight of the cleansing composition. In certain embodiments, the first surfactant can be present in an amount of from about 10% to about 50%, about 20% to about 30%, or about 25% to about 35% by weight, based on the total weight of the cleansing composition. In particular embodiments, the first surfactant can be present in an amount of about 10%, about 15%, about 20%, about 25%, about 30%, about 35%, about 40%, about 45%, or about 50% by weight, based on the total weight of the cleansing composition. In certain embodiments, the second surfactant can be present in an amount of at least about 15%, at least about 20%, at least about 25%, or at least about 30% by weight, based on the total weight of the cleansing composition. In certain embodiments, the second surfactant can be present in an amount of from about 10% to about 50%, about 20% to about 30%, or about 25% to about 35% by weight, based on the total weight of the cleansing composition. In particular embodiments, the second surfactant can be present in an amount of about 10%, about 15%, about 20%, about 25%, about 30%, about 35%, about 40%, about 45%, or about 50% by weight, based on the total weight of the cleansing composition. The first surfactant and second surfactant may be present in a weight ratio of from about 0.8:1 to about 10:1, about 1:1 to about 8:1, or about 1:1 to about 5:1, respectively. In certain embodiments, the first surfactant and the second surfactant may be present in a weight ratio of about 0.8:1, about 1:1, about 1:1.5, about 5:1, about 8:1, or about 10:1, respectively.

In certain embodiments, the first surfactant can be in an amount equal to or greater than the second surfactant. In particular embodiments, the first surfactant and the second surfactant can both be anionic surfactants, e.g., the first surfactant can be sodium methyl cocoyl taurate and the second surfactant can be sodium cocoyl isethionate. In certain embodiments, the cleansing composition can include sodium methyl cocoyl taurate and sodium cocoyl isethionate present in a weight ratio of from about 0.8:1 to about 10:1, respectively. It was surprisingly and advantageously found that sodium methyl cocoyl taurate as the first surfactant present in an amount equal to or greater than the sodium cocoyl isethionate as the second surfactant provided for increased solubility. Improved solubility was also achieved during manufacturing in which sodium cocoyl isethionate was added to sodium methyl cocoyl taurate during the blending process compared to the addition of sodium methyl cocoyl taurate to sodium cocoyl isethionate.

### 4.2.2. Chelating Agents

The cleansing composition can include one or more chelating agents. Chelating agents suitable for use with the present disclosure include disodium ethylenediaminetetraacetic acid (EDTA), tetrasodium glutamate diacetate, sodium citrate, citric acid, or combinations thereof. In certain embodiments, the chelating agent can be disodium EDTA. A person skilled in the art will appreciate a wide variety of chelating agents are suitable for use with the present disclosure.

In certain embodiments, the one or more chelating agents can be present in an amount of from about 1% to about 15%, about 1% to about 10%, or about 1% to about 5% by weight, based on the total weight of the cleansing composition. In particular embodiments, the one or more chelating agents can be present in an amount of about 1%, about 3%, about 5%, about 8%, about 10%, or about 15% by weight, based on the total weight of the cleansing composition.

### 4.2.3. pH Adjusters

The cleansing composition can include one or more pH adjusters. pH adjusters suitable for use with the present disclosure include, for example, citric acid, magnesium sulfate, monopotassium phosphate, dipotassium phosphate, sodium hydroxide, sodium bicarbonate, sodium citrate, or combinations thereof. In certain aspects, the one or more pH adjusters can be acidic in nature. In certain aspects, the one or more pH adjusters can be basic in nature. In certain embodiments, the one or more pH adjusters can include citric acid, magnesium sulfate, or combinations thereof. In particular embodiments, the cleansing composition can include a combination of citric acid and magnesium sulfate. A person skilled in the art will appreciate a wide variety of buffering agents are suitable for use with the present disclosure.

In certain embodiments, the one or more pH adjusters can be present in an amount of from about 1% to about 25%, about 5% to about 20%, or from about 5% to about 10% by weight, based on the total weight of the cleansing composition. In particular embodiments, the one or more buffering agents can be present in an amount of about 1%, about 5%, about 7%, about 10%, about 14%, about 15%, about 20%, or about 25% by weight, based on the total weight of the cleansing composition.

### 4.2.4. Preservatives

The cleansing composition can include one or more preservatives. A person skilled in the art will appreciate a wide variety of preservatives are suitable for use with the present disclosure. In certain embodiments, the one or more preservatives can include sodium benzoate, potassium sorbate, p-anisic acid, sodium dehydroacetate, benzoic acid, or combinations thereof. In particular embodiments, the one or more preservatives can include sodium benzoate.

In certain embodiments, the one or more preservatives can be present in an amount of from about 1% to about 30%, about 5% to about 25%, or about 15% to about 25% by weight, based on the total weight of the cleansing composition. In particular embodiments, the one or more preservatives can be present in an amount of about 1%, about 5%, about 10%, about 15%, about 20%, about 25%, or about 30% by weight, based on the total weight of the cleansing composition.

### 4.2.5. Antioxidants

The cleansing composition can include one or more antioxidants. A person skilled in the art will appreciate a wide variety of antioxidants are suitable for use with the present disclosure. In certain embodiments, the one or more antioxidants can include ascorbic acid, sodium ascorbyl phosphate, ascorbyl palmitate, tocopherol, ascorbyl glucoside, ascorbyl tetraisopalmitate, or combinations thereof. In certain embodiments, the one or more antioxidants can include ascorbic acid. In certain embodiments, the one or more antioxidants can include sodium ascorbyl phosphate.

In certain embodiments, the one or more antioxidants can be present in an amount of from about 0.1% to about 5%, about 0.5% to about 3%, or about 1% to about 2%. In particular embodiments, the one or more antioxidants can be present in an amount of about 0.1%, about 0.5%, about 1%, about 1.5%, about 2%, about 3%, or about 5% by weight, based on the total weight of the cleansing composition.

### 4.2.6. Additional Additives

The cleansing composition can include one or more additional additives. In certain aspects, the one or more additional additives can include one or more colorants, one or more fragrances, one or more bulking/anti-caking agents, one or more skin benefit agents, one or more effervescent agents, one or more thickeners, and combinations thereof. A person skilled in the art will appreciate a wide variety of additional additives are suitable for use with the present disclosure.

### Colorants

The cleansing composition can include one or more colorants. A person skilled in the art will appreciate a wide variety of colorants are suitable for use with the present disclosure. In certain embodiments, the one or more colorants can include organic dyes, mineral pigments, or combinations thereof. In certain embodiments, the cleansing composition can be substantially free of added colorants.

The cleansing composition can include the one or more colorants in an amount of from about 0.1% to about 3%, about 0.5% to about 2%, or about 1% to about 1.5% by weight, based on the total weight of the cleansing composition. In certain embodiments, the cleansing composition can include the one or more colorants in an amount of about 0.1%, about 0.5%, about 1%, about 1.5%, about 2%, about 2.5%, or about 3% by weight, based on the total weight of the cleansing composition. In particular embodiments, the cleansing composition can include about 3% or less, about 2% or less, about 1.5% or less, about 1% or less, or about 0.5% or less by weight of the one or more colorants, based on the total weight of the cleansing composition.

### Fragrances

The cleansing composition can include one or more fragrances. A person skilled in the art will appreciate a wide variety of fragrances are suitable for use with the present disclosure. In certain embodiments, the one or more fragrances can include individual ingredients or a mixture of ingredients with olfactive properties. In certain embodiments, the cleansing composition can be substantially free of added fragrances.

The cleansing composition can include the one or more fragrances in an amount of from about 0.01% to about 1%, about 0.05% to about 0.8%, or about 0.1% to about 0.5% by weight, based on the total weight of the cleansing composition. In certain embodiments, the cleansing composition can include the one or more fragrances in an amount of about 0.01%, about 0.02%, about 0.05%, about 0.08%, about 0.1%, about 0.3%, about 0.4%, about 0.5%, about 0.6%, about 0.7%, about 0.8%, about 0.9%, or about 1% by weight, based on the total weight of the cleansing composition. In particular embodiments, the cleansing composition can include about 1% or less, about 0.8% or less, about 0.5% or less, about 0.4% or less, about 0.3% or less, about 0.1% or less, about 0.08% or less, or about 0.05% or less of the one or more fragrances, based on the total weight of the cleansing composition.

### Bulking/Anti-Caking Agents

The cleansing composition can include one or more bulking/anti-caking agents. The one or more bulking/anti-caking agents can reduce or prevent the formation of agglomerated materials in a powder and improve flow during formation of a final product, as well as for easing packaging, transport, and consumption. Certain bulking/anti-caking agents are soluble in water while others are soluble in alcohols or other organic solvents. In certain aspects, bulking/anti-caking agents can function by either absorbing excess moisture or by coating particles thereby allowing them to repel water more effectively. Suitable bulking/anti-caking agents for use in the present disclosure include, for example, celluloses, starches, silica and silicates, and metal salts. In certain embodiments, the one or more bulking/anti-caking agents can include silica, calcium silicate, corn starch, aluminum starch octenylsuccinate, hydroxypropyl methylcellulose, sodium bicarbonate, or combinations thereof. A person skilled in the art will appreciate a wide variety of bulking/anti-caking agents are suitable for use with the present disclosure.

The cleansing composition can include the one or more bulking/anti-caking agents in an amount of from about 1% to about 20%, about 5% to about 15%, or about 10% to about 18% by weight, based on the total weight of the cleansing composition. In certain embodiments, the cleansing composition can include the one or more bulking/anti-caking agents in an amount of about 1%, about 5%, about 10%, about 15%, about 18%, or about 20% by weight, based on the total weight of the cleansing composition.

### Skin Benefit Agents

The cleansing composition can include one or more active agents providing one or more skin benefits/ingredients. For example, and not by way of limitation, the cleansing composition can include one or more skin benefit agents including one or more of anti-acne agents, anti-aging agents, anti-microbial agents, or combinations thereof. In certain aspects, such skin benefit agents can include, for example, one or more of: retinol, retinyl esters, tetronic acid, tetronic acid derivatives, hydroquinone, kojic acid, gallic acid, arbutin, ct-hydroxy acids, niacinamide, pyridoxine, ascorbic acid, vitamin E and derivatives, aloe, salicylic acid, benzoyl peroxide, witch hazel, caffeine, zinc pyrithione, fatty acid esters of ascorbic acid, colloidal oatmeal, acids such as alpha hydroxy acid, polyhydroxy acid, and beta hydroxy acid, enzymes such as chlorella, papaya, and papain, N-acetylglucosamine, gluconolactone, willow bark extract, hyaluronic acid, oat extract, aloe extract, and amino acids.

In certain aspects, the one or more skin benefit agents can include one or more emulsifiers, one or more silicones, or combinations thereof. The one or more emulsifiers can include potassium cetyl phosphate. The one or more silicones can include dimethicone, cyclomethicone, amodimethicone, or combinations thereof. A person skilled in the art will appreciate a wide variety of skin benefit ingredients/carriers are suitable for use with the present disclosure.

The one or more skin benefit agents can be present in an amount of from about 1% to about 30%, about 5% to about 25%, or about 2% to about 5% by weight, based on the total weight of the cleansing composition. In certain embodiments, the one or more skin benefit agents can be present in an amount of about 1%, about 2%, about 5%, about 7%, about 10%, about 15%, about 20%, about 25%, or about 30% by weight, based on the total weight of the cleansing composition.

### Effervescent Agents

The cleansing composition can include one or more effervescent agents. A person skilled in the art will appreciate a wide variety of effervescent agents are suitable for use with the present disclosure. For example, and not by way of limitation, the one or more effervescent agents can include sodium bicarbonate, citric acid, and combinations thereof. In certain embodiments, the one or more effervescent agents can include sodium bicarbonate and citric acid.

The one or more effervescent agents can be present in an amount of from about 1% to about 60%, about 10% to about 45%, or about 15% to about 30% by weight, based on the total weight of the cleansing composition. In certain embodiments, the one or more effervescent agents can be present in an amount of about 5%, about 10%, about 20%, about 30%, about 45%, about 50%, or about 60% by weight, based on the total weight of the cleansing compositions. In certain embodiments, the cleansing composition can include two or more effervescent agents. The first effervescent agent can be present in an amount of from about 1% to about 30%, about 5% to about 20%, or about 10% to about 15% by weight, based on the total weight of the cleansing composition. The second effervescent agent can be present in an amount of from about 1% to about 30%, about 5% to about 20%, or about 10% to about 15% by weight, based on the total weight of the cleansing composition. In certain embodiments, the first effervescent agent can be sodium bicarbonate and the second effervescent agent can be citric acid.

### Thickeners

The cleansing composition can include one or more thickeners. A person skilled in the art will appreciate a wide variety of thickeners are suitable for use with the present disclosure. In certain embodiments, the one or more thickener can include xanthan gum, carrageenan, acrylates, cross-polymers, carbomers, celluloses, or combinations thereof.

The one or more thickeners can be present in an amount of from about 1% to about 10%, about 3% to about 8%, or about 1% to about 5% by weight, based on the total weight of the cleansing composition. In certain embodiments, the one or more thickeners can be present in an amount of about 1%, about 3%, about 5%, about 8%, or about 10% by weight, based on the total weight of the cleansing composition.

### 4.2.7. Formulations

In certain embodiments, the cleansing composition can include one or more surfactants, one or more chelating agents, one or more pH adjusters, one or more preservatives, one or more antioxidants, or combinations thereof. The cleansing composition can include from about 60% to about 90% by weight of the one or more surfactants; about 1% to about 15% by weight of the one or more chelating agents; about 1% to about 25% by weight of the one or more pH adjusters; about 1% to about 30% by weight of the one or more preservatives; and about 0.1% to about 5% by weight of the one or more antioxidants, based on the total weight of the cleansing composition. In certain embodiments, the one or more surfactants can include at least two anionic surfactants, e.g., sodium methyl cocoyl taurate and sodium cocoyl isethionate. The one or more chelating agents can include disodium EDTA. The one or more pH adjusters can include citric acid and magnesium sulfate. The one or more preservatives can include sodium benzoate. The one or more antioxidants can include ascorbic acid. In certain embodiments, the one or more antioxidants can include sodium ascorbyl phosphate.

### 4.3. Methods of Making Cleansing Compositions

The present disclosure provides methods of making cleansing compositions. In certain embodiments, the components of the cleansing composition can be in the form of a powder. If necessary, the method of forming the cleansing composition can include an initial step of rolling or milling individual components into the form of a powder. For example, the first step may be to roll or mill the one or more surfactants into a powder. By way of example, a first surfactant may initially be in the form of flakes and the second surfactant may be in the form of noodles, but each of these surfactants are milled (either together or individually) to form a powder having a desired particle size.

When making a cleansing product described herein, the surfactant (or surfactants, if used) are added to a dry powder blender. To the surfactant(s) is added a mixture of other dry components. For example, the mixture of other dry components may include the chelating agent(s), bulking agent(s), buffering agent(s), anti-caking agent(s), and other optional components (such as anti-acne agents, colorants, fragrances). These components are then mixed in the blender for a sufficient time to achieve homogeneity. In certain aspects, a uniform flowable powder can be formed. Once mixed, the final product has been prepared.

The final product, whether in the form of a loose powder, or in any other form, can be packaged in a suitable container. The container can be substantially air-tight or fully air-tight. In certain embodiments, the final product can be packaged in a laminate sachet. Once the cleansing product is formed and packaged, it can be distributed to a user or users to provide at least a cleansing function during use. In certain aspects, the final product can be in the form of a kit including the solid cleansing composition in suitable packaging such as a laminate sachet and a container suitable for containing a liquid such as water.

### 4.4. Methods of Using Cleansing Compositions

Cleansing compositions of the present disclosure can be used, for example, in combination with a liquid (e.g., water) such that the cleansing composition (e.g., in a powder form) fully dissolves in the liquid prior to application to the skin of a user. In certain aspects, cleansing compositions of the present disclosure can fully dissolve in a liquid such as water in about 2 minutes or less, about 1 minute or less, or about 30 seconds or less. Any suitable type of clean water is suitable for use with the cleansing compositions of the present disclosure. In certain embodiments, the water is of drinkable quality. Any drinking quality water is suitable for use with the present disclosure, for example, and not by way of limitation, tap water, filtered water, purified water, residential well water, or bottled water.

Once dispensed in the form of a powder, the user can add the cleansing composition to an amount of liquid such as water (e.g., about 200-250 mL) to form a wetted cleansing product, and run the wetted cleansing product within the user's hands, fingers, body, or face. In certain aspects, the cleansing product can be added to an amount of liquid such as water and shaken for about 1-2 minutes to form the wetted product. The cleansing composition and the liquid can be combined in any suitable container, for example, an aluminum bottle with a foaming pump. For example, and not by way of limitation, the cap of the container can include a cap, flip-top cap, a pump, or a self-foaming pump. By running the wetted product within the user's hands, fingers, body, or face, the wetted product performs a cleansing function. In certain aspect, the wetted cleansing product can have foaming properties and can be in the form of a gentle foam when applied to a user's skin. When cleansing is complete, the user may rinse the applied wetted cleansing product with water. In certain embodiments, the user can add the cleansing composition to an amount of liquid such that the wetted cleansing product includes from about 1% to about 20% or about 3% to about 10% by weight of the cleansing composition and from about 80% to about 99% or about 90% to about 97% by weight liquid, based on the total weight of the wetted cleansing product. In particular embodiments, the final product can include about 3% by weight of the cleansing composition and about 97% by weight liquid, based on the total weight of the wetted cleansing product.

### 4.5. Features of Cleansing Compositions

The present disclosure provides improved cleansing compositions. Such cleansing compositions can be in a solid form, e.g., a powder form including a loose powder, crystalline powder, or granules. When in such form, the components of the cleaning composition can be subjected to milling or rolling to provide the desired form. The cleansing compositions of the present disclosure are suitable for use directly on the user's skin or face.

The cleansing compositions herein may be packaged in any desired method or packaging, which may be dependent upon the particular form of the cleansing product. For the form of a flowable powder, the cleansing product may be stored in a single-use, air-tight package or sachet, or may be stored in a container (such as a tube, canister, bottle, or ampoule) whereby a desired amount of cleansing product can be dispensed by the user as needed. Thus, the cleansing product can be stored in a multi-use container, where the user takes a desired amount of product for use, or the product can be stored in single-use containers, whereby the package dispenses a single amount of cleansing product upon opening the container.

The cleansing product can be packaged in a substantially water-tight package and an air-tight package. For example, the product can be contained within its own single-use packaging, such as a sachet, a capsule, a dissolvable film, a blister package, or other single-wrapped or sealed environment. The user releases the cleansing product from its packaging for use, such as by unwrapping or releasing it from a blister package, or separating/breaking a capsule, or dissolving an outer film. In aspects where the cleansing product is in the form of a powder, the user may not need to manipulate the cleansing product to render it more broken apart.

The cleansing product may be encapsulated in an outer capsule or other shell, may be encapsulated within an outer dissolvable film or casing, where the outer material may be either separated or broken to release the cleansing product, or dissolved during use, thereby releasing the cleansing components contained therein. In some aspects, the cleansing product is a granulated or powdered material, which may be dispensed from a storage container prior to use.

### 5. EXAMPLES

The following Examples are intended to illustrate, but not to limit, the disclosed subject matter in any manner, shape, or form, either explicitly or implicitly.

### Example 1: Solid Cleansing Compositions

The present Example provides exemplary solid cleansing compositions, as shown in Tables 1 and 2 (below), in accordance with the present disclosure. One composition (Table 1, Formulation 1) included two surfactants of anionic nature (i.e., sodium methyl cocoyl taurate and sodium cocoyl isethionate) in a combined weight of about 61% by weight, based on the total weight of the solid cleansing composition. The two surfactants were present in a weight amount approximately equal to each other. Sodium methyl cocoyl taurate was present in a greater amount (32.5% by weight) than sodium cocoyl isethionate (28.5% by weight). The composition further included a chelating agent (disodium EDTA), a preservative (sodium benzoate), two pH adjusters (magnesium sulfate and citric acid), and an antioxidant (ascorbic acid). The composition was in the form of a powder.

TABLE 1
Formulation 1

| Component | Function | Amount (wt %) |
|---|---|---|
| Sodium Methyl Cocoyl Taurate | Surfactant | 32.5 |
| Sodium Cocoyl Isethionate | Surfactant | 28.5 |
| Disodium EDTA | Chelating Agent | 3.0 |
| Sodium Benzoate | Preservative | 20.0 |
| Magnesium Sulfate | pH Adjuster | 8.0 |
| Citric Acid | pH Adjuster | 7.0 |
| Ascorbic Acid | Antioxidant | 1.0 |

| | |
|---|---|
| Total | 100.0 |

The second composition (Table 2, Formulation 2) included two surfactants of anionic nature (i.e., sodium methyl cocoyl taurate and sodium cocoyl isethionate) in a combined weight of about 61% by weight, based on the total weight of the solid cleansing composition. The two surfactants were present in a weight amount approximately equal to each other. Sodium methyl cocoyl taurate was present in a greater amount (32.5% by weight) than sodium cocoyl isethionate (28.5% by weight). The composition further included a chelating agent (disodium EDTA), a preservative (sodium benzoate), two pH adjusters (magnesium sulfate and citric acid), and an antioxidant (sodium ascorbyl phosphate). The composition was in the form of a powder.

TABLE 2
Formulation 2

| Component | Function | Amount (wt %) |
|---|---|---|
| Sodium Methyl Cocoyl Taurate | Surfactant | 32.5 |
| Sodium Cocoyl Isethionate | Surfactant | 28.5 |
| Disodium EDTA | Chelating Agent | 3.0 |
| Sodium Benzoate | Preservative | 20.0 |
| Magnesium Sulfate | pH Adjuster | 7.0 |
| Citric Acid | pH Adjuster | 7.0 |
| Sodium Ascorbyl Phosphate | Antioxidant | 2.0 |

| | |
|---|---|
| Total | 100.0 |

### Example 2: Manufacturing Filling

The present Example provides Formulations 3-6 (Table 3). All formulations failed manufacturing filling either by predictive models or at the manufacturing site. It was determined that liquid cannot be used in the final formulation.

TABLE 3

| | | Formu- | Formu- | Formu- | Formu- |
|---|---|---|---|---|---|
| | | lation 3 | lation 4 | lation 5 | lation 6 |
| Sodium Methyl | Surfactant | 29.8 | 36.3 | 36.3 | 36.3 |
| Cocoyl Taurate | | | | | |
| Sodium Cocoyl | Surfactant | 29.5 | 30.0 | 29.0 | 29.0 |
| Isethionate | | | | | |
| Disodium EDTA | Chelating | 5.0 | 5.0 | 5.0 | 6.0 |
| | Agent | | | | |
| Sodium | Preservative | 16.7 | 16.7 | 16.7 | 16.7 |
| Benzoate | | | | | |
| Magnesium | pH Adjuster | 6.0 | 6.0 | 9.0 | 10.0 |
| Sulfate | | | | | |
| Phenoxyethanol | Preservative | 13.0 | 6.0 | 4.0 | 2.0 |
| Citric Acid | pH Adjuster | 0.0 | 0.0 | 0.0 | 0.0 |
| Total | | 100.0 | 100.0 | 100.0 | 100.0 |

### Example 3: Preservative Systems/Microbial Testing

The present Example provides preservative systems evaluated and failed microbial testing.

TABLE 4

| | Conventional Preservatives | | | | Preservative Boosters | | |
|---|---|---|---|---|---|---|---|
| | pH dependent | | pH independent | | pH independent | | |
| Reconstituted | Sodium | Potassium | | Phenoxy- | Caprylyl | Ethylhexyl | |
| in water at | Benzoate | Sorbate | Chlorphenesin | ethanol | Glycol | glycerin | pH |
| 2% | | | 0.1 | 0.2 | | | |
| 2% | | | 0.1 | 0.2 | | | |
| 3% | | | 0.15 | 0.3 | | | 5.78 |
| 3% | | | 0.15 | 0.3 | 0.18 | | 5.7 |
| 3% | | 0.3 | | | | 0.33 | 6.05 |

### Example 4: Manufacturing Filling/Microbial Testing

The present Example provides Formulations 7-10 (Table 5). All samples were successful at manufacturing filling, but failed microbial testing when reconstituted with hard (high pH) water.

TABLE 5

| | Formu- | Formu- | Formu- | Formu- | Formu- |
|---|---|---|---|---|---|
| | lation 7 | lation 8 | lation 9 | lation 10 | lation 11 |
| Sodium Methyl | Surfactant | 37.3 | 34.0 | 35.0 | 34.7 |
| Cocoyl Taurate | | | | | |
| Sodium Cocoyl | Surfactant | 31.0 | 29.0 | 29.0 | 29.0 |
| Isethionate | | | | | |
| Disodium | Chelating | 5.0 | 5.0 | 5.0 | 5.0 |
| EDTA | Agent | | | | |
| Sodium | Preservative | 16.7 | 20.0 | 20.0 | 20.0 |
| Benzoate | | | | | |
| Magnesium | pH Adjuster | 10.0 | 12.0 | 10.00 | 10.0 |
| Sulfate | | | | | |
| Citric Acid | pH Adjuster | 0.0 | 0.0 | 1.0 | 1.3 |
| Total | | 100.0 | 100.0 | 100.0 | 100.0 |

While systems and methods have been described in connection with the various embodiments of the description, it will be appreciated by those skilled in the art that changes could be made to the embodiments without departing from the broad inventive concept thereof. It is understood, therefore, that this disclosure is not limited to the particular embodiments disclosed, and it is intended to cover modifications within the spirit and scope of the present disclosure as defined by the claims.

It is to be appreciated that certain features of the invention which are, for clarity, described herein in the context of separate embodiments, may also be provided in combination in a single embodiment. That is, unless obviously incompatible or specifically excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. Finally, while an embodiment may be described as part of a series of steps or part of a more general structure, each said step may also be considered an independent embodiment in itself, combinable with others.

It should be understood that the steps of the exemplary methods set forth herein are not necessarily required to be performed in the order described, and the order of the steps of such methods should be understood to be merely exemplary. Likewise, additional steps may be included in such methods, and certain steps may be omitted or combined, in methods consistent with various embodiments of the present invention. Although the elements in the following method claims, if any, are recited in a particular sequence with corresponding labeling, unless the claim recitations otherwise imply a particular sequence for implementing some or all of those elements, those elements are not necessarily intended to be limited to being implemented in that particular sequence.

1. A solid cleansing composition, comprising:
a. at least a first surfactant present in a first surfactant weight amount; and
b. at least a second surfactant present in a second surfactant weight amount, wherein the combined weight of the first surfactant and the second surfactant is at least about 60% by weight, based on the total weight of the solid cleansing composition, and wherein the solid cleansing composition is in a powder form.

2. The solid cleansing composition of claim 1, wherein the combined weight of the first surfactant and the second surfactant is from about 60% to about 90% by weight, based on the total weight of the solid cleansing composition.

3. The solid cleansing composition of claim 1, wherein the first surfactant weight amount and the second surfactant weight amount are approximately equal to each other.

4. The solid cleansing composition of claim 1, wherein the first surfactant and the second surfactant have the same ionic nature.

5. The solid cleansing composition of claim 4, wherein the first surfactant and the second surfactant are selected from anionic surfactants, nonionic surfactants, cationic surfactants, or amphoteric surfactants.

6. The solid cleansing composition of claim 1, wherein the first surfactant is a taurate.

7. The solid cleansing composition of claim 1, wherein the second surfactant is an isethionate.

8. The solid cleansing composition of claim 1, wherein the solid cleansing composition is substantially free of sulfate surfactants.

9. The solid cleansing composition of claim 1, wherein the solid cleansing composition further comprises one or more pH adjusters comprising citric acid, magnesium sulfate, or combinations thereof.

10. The solid cleansing composition of claim 1, wherein the solid cleansing composition is dissolvable in a liquid.

11. A kit, comprising:
a. a solid cleansing composition, comprising:
i. at least a first surfactant; and
ii. at least a second surfactant,
wherein the combined weight of the first surfactant and the second surfactant is at least about 60% by weight, based on the total weight of the solid cleansing composition, and
wherein the solid cleansing composition is in powder form; and
b. a container configured to contain a liquid.

12. A cleansing product, comprising:
a. from about 2% to about 20% by weight of a solid cleansing composition, based on the total weight of the cleansing product, the solid cleansing composition comprising:
i. at least a first surfactant; and
ii. at least a second surfactant,
wherein the combined weight of the first surfactant and the second surfactant is at least about 60% by weight, based on the total weight of the solid cleansing composition, and wherein the solid cleansing composition is in powder form; and
b. from about 80% to about 98% by weight of a liquid, based on the total weight of the cleansing product, wherein the cleansing composition is dissolved in the liquid.
